(19) United States
(12) Patent Application Publication
(10) Pub. No.: US 2010/0175139 A1
(43) Pub. Date: Jul. 8, 2010

(54) SOMATOTRANSGENIC BIOIMAGING

(76) Inventors: Simon Nicholas Waddington, London (GB); Tristan Rowntree McKay, Manchester (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/531,050

(22) PCT Filed: Mar. 13, 2008
(86) PCT No.: PCT/GB2008/000877

§ 371 Date: Mar. 10, 2010

(30) Foreign Application Priority Data
Mar. 13, 2007 (GB) 0704828.3
Mar. 13, 2007 (GB) 0704829.1

(51) Int. Cl. G01N 33/00; A01K 67/00

(52) U.S. Cl. 800/3; 800/9

(57) ABSTRACT

The invention relates to modelling diseases, to screening for compounds that modulate such diseases and to as-saying drug metabolism and toxicity in non-human transgenic animals, by a novel technique developed by the inventors known as “somatotransgenic bioimaging”. The invention thus provides: a method for determining whether the expression of a reporter gene is modulated by a compound or a method of evaluating the metabolism and/or toxicity of a compound, said method comprising: (a) administering said compound to a non-human transgenic animal, generated by gene transduction of one or more specific tissues when in utero or neonatal, with a vector comprising a bioluminescent reporter gene operably linked to a genetic element responsive to a pathology or therapy or to a genetic element responsive to drug metabolism and/toxicity; and (b) determining whether or not of said compound has an effect on the expression of said reporter gene in said specific tissue or tissues and/or determining the extent of any such effect, said determination comprising detecting from the animal bioluminescence caused by the activity of the gene product of the reporter gene. In some embodiments cells pre-transduced with vectors of the invention may also be introduced into the animals instead of delivering the vectors directly.

### FIELD OF THE INVENTION

The invention relates to modelling pathologies, screening for compounds that modulate such pathologies and to evaluating drug metabolism and toxicity in non-human transgenic animals by a novel technique termed “somatotransgenic bioimaging”.

### BACKGROUND OF THE INVENTION

### Drug Validation

Potential therapeutics are generally identified using high-throughput in vitro technologies to begin with. It is then desirable to validate successful candidate compounds in vivo, for example in rodent models, before progressing to full-scale pre-clinical primate studies or clinical trials.

Traditional pharmacological assays rely on taking measurements from peripheral, secreted or excreted body fluids or tissue biopsies and often rely on endpoint analyses. Measurements from fluids or tissues rely on an appropriate experimental variable, i.e. they can only work if there is something in the fluid or tissue that changes in response to the administration of the candidate compound. Endpoint analyses rely on sacrifice of animals, which perturbs the experimental continuum, necessitating large cohorts to provide reliable statistical analysis. The advent of transgenic mice has revolutionised the drug validation process by providing genetically engineered disease models. The field of transgenic disease modelling has recently progressed to the generation of mice transgenic for the luciferase reporter gene under the control of a tissue or phenotype specific promoter (W. Zhang et al. 2001, Transgenic Research 10:423). High fidelity bioimaging permits the investigator to follow the genetic activation (or repression) of a specific drug target quantitatively in vivo over the lifetime of the animal.

By its very nature, a standard transgenic animal obtained by germline transgenesis contains the inserted genetic material in every cell of its body. Most intracellular signalling processes are common between the different organ systems within the body and, significantly, may have contrasting effects in different tissues. Such activity over the whole body causes significant and complex background interference during imaging which impedes the use of such transgenics for effective, continual bioimaging. In such instances, investigators will resort to endpoint analysis of individual post-mortem tissues. The present invention addresses these issues.

### Evaluation of Drug Metabolism and Toxicity

Drug metabolism is the major determinant of drug clearance and inducible expression of drug-metabolising cytochrome P450s (CYPs) is the factor most frequently responsible for variable pharmacokinetics. These haem-containing enzymes play a key role in the metabolism (mainly oxidation) of a variety of chemically diverse compounds including food compounds, pharmaceutical agents, carcinogens, and environmental pollutants.

Two procedures are commonly used for in vitro investigation of the metabolic profile of a drug: incubation with liver microsomes and incubation with metabolically competent cells.

The metabolic stability of a drug in liver microsomes of different species is determined in order to assess the potential of this compound to form undesired potentially toxic or pharmacologically inactive metabolites due to phase I metabolism or to accumulate in the body due to lacking or negligible metabolic degradation. The determination of the metabolic stability is therefore a measure to describe the metabolic fate. The determination of the metabolic stability in liver microsomes summarizes all the possible reactions. Liver microsomes are subcellular fractions (mainly endoplasmatic reticulum) containing many drug-metabolizing enzymes, including CYPs. Therefore they are widely used as an in vitro model system in order to investigate the metabolic fate of xenobiotics. Human liver microsomes contain the following CYP isoenzymes involved in drug metabolism: CYP1A2, 2A6, 2B6, 2C8, 2C9, 2C19, 2D6, 2E1, and 3A4. Of these isoenzymes CYP3A4 plays a major role in metabolism of xenobiotics as it is the most abundant CYP in human liver (approx. 28%) and it is involved in metabolism of more than 50% of all pharmaceuticals applied in present-day medication.

The major limitation of microsomes is that they express phase I activities, but only part of phase II activities, and can only be used for short incubation times.

When intact cells are used, gene expression, metabolic pathways, cofactors/enzymes and plasma membrane are largely preserved, but fully differentiated cells such as primary cultured hepatocytes need to be used, since hepatoma cell lines have only very low and partial CYP expression.

Inhibition of CYP is an undesirable feature for a drug candidate, and needs to be addressed by examining whether the drug candidate inhibits the metabolism of other compounds or whether other compounds inhibit the metabolism of the drug candidate. Such experiments can be conducted both with microsomes and in cells. The major limitation of microsomes is that inhibition parameters may not accurately reflect the situation in vivo, since the contribution of drug transport is not considered. The best picture of a potential drug-drug interaction can be obtained in metabolically competent hepatocytes. This requires the use of a cellular system fully capable of transcribing and translating CYP genes, and can be monitored in vitro as an increase in enzyme mRNA or activity. Human hepatocytes in primary culture respond well to enzyme inducers during the first few days; this ability is lost thereafter. Hepatoma cell lines respond poorly to inducers, although the induction of a few isoenzymes has been reported. Primary cultured hepatocytes are still the unique in vitro model that allows global examination of the CYP-inductive potential of a drug.

Potential therapeutics are generally identified using high-throughput in vitro technologies to begin with. It is then desirable to validate successful candidate compounds in vivo, for example in rodent models, before progressing to full-scale pre-clinical primate studies or clinical trials. Currently there are few reliable in vivo assay systems for the analysis of CYP activation due to drug metabolism.

The Hepatic Reductase Null (HRN) mouse developed by CXR Biosciences is one current model for measuring the effect that CYPs play on the metabolism of candidate drugs. In order to function, CYPs receive electrons from electron donor Cytochrome P450 reductase. In the HRN mouse this reductase activity is knocked out thereby preventing the activity of any CYPs. This provides a model system negating CYP activity allowing clearer analysis of alternative metabolites or providing efficacy drug analyses without CYP metabolism. The uses are limited in the context of delineating specific CYP activity in drug metabolism.

Alternative in vivo strategies involve generating mice with “humanised” livers. This negates the disparity between relative human and rodent CYP activity in response to drug administration. Tateno et al. (2004) (Tateno C. et al. Am J Pathol; 165: 3 p901) describe a process by which uPA/SCID mice can undergo partial ablation of the hosts hepatocytes followed by reconstitution with human hepatocytes to create a humanised mouse. This process has actually been recently used by Katoh et al. (2007) (Katoh, M. et al. J Pharm Sci; 96: 2, p428) to assay CYP2D6 specific metabolites in these humanised mice in response to high or low levels of human albumin. Although this work shows elegant proof of concept, it relies on CYP inhibitors which are not specific amongst polymorphic isoenzymes and depends on the secretion of quantifiable metabolic products in the serum. Peripheral blood sampling and biochemical analysis is time consuming and finite under animal experimental guidelines.

The present invention addresses such issues by using genetic elements well characterised in the current literature to model upregulation of metabolism of target drugs in vivo. The process is rapid and malleable in that somatotransgenics for particular CYP450s can be generated within weeks. Readout is in real-time which allows measurements within individual animals to be made before, during and after drug administration. Furthermore, application and readout can be made on top of any knockout, phenotype or disease model without having to carry out time consuming mating crosses. Our technology facilitates targeting of vector to the liver and so any bioimaging readout is restricted to the organ of choice.

### SUMMARY OF THE INVENTION

We have developed a novel technique known as “somatotransgenic bioimaging”. In this technique, vectors carry a bioluminescent reporter gene driven by pathology or therapy responsive genetic elements that model progression of the pathology and/or therapeutic intervention. The vector is delivered to a non-human foetal or neonatal animal via targeted administration to a specific tissue or tissues and the animal is allowed to mature. The expression of the bioluminescent reporter gene can be measured in the intact, living animal. Any number of measurements can therefore be taken using the same animal without the need for sacrifice of the animal. Measurements can be taken for example in response to disease defined molecular events. As another example, measurements can be taken prior to drug administration, to obtain steady state data and then from drug application through complete metabolism of the drug until steady state is once again attained.

This technique is useful, for example, for determining whether a compound modulates the expression of a gene that controls the development of a disease. It is therefore useful in the validation of candidate drug compounds.

This technique is also useful, for example, for determining whether a compound modulates the expression of a gene that controls the metabolism of or toxic responses due to a drug administration. The primary class of drug metabolizers alluded to is the cytochrome P450 (CYP) enzymes. Cytochrome P450 enzymes are the major catalysts for the oxidative metabolism of a vast array of compounds. Metabolism of drugs by CYPs influences drug clearance, toxicity, activation and, potentially, adverse interactions with other drugs. Compounds that are turned over and cleared from the body rapidly or that are converted to toxic products by P450 enzymes may be poor drug candidates. Drugs that induce or suppress expression of a P450 enzyme can also have a deleterious effect on the efficacy or toxicity of a second drug. According to the invention, it is possible to place a bioluminescent reporter gene under the control of a genetic element that controls the expression of a metabolic enzyme such as a cytochrome P450 enzyme, target the construct to the liver of an animal by in utero gene transfer, and determine the effect of a compound on the expression of the metabolic enzyme indirectly by monitoring the expression of the reporter gene using whole animal bio-imaging. The invention is therefore useful for determining the potential speed of clearance and hence likely efficacy of a drug candidate, its toxicity and likely effect on efficacy or toxicity of other drugs that are to be co-administered.

We have previously demonstrated efficient gene delivery and persistent transgene expression by lentiviral gene delivery to the foetal rodent via the vitelline vessels (S. N. Waddington et al. 2003, Gene Therapy 10:1234). In this paper, high-dose attenuated VSV-G pseudotyped equine infectious anaemia virus (EIAV) encoding β-galactosidase under the control of the CMV promoter was injected into the foetal circulation of immuno-competent MF1 mice. Efficient gene delivery and persistent transgene expression indicated a potential for the technique in gene therapy. This technique of in utero gene delivery was further investigated to determine whether it would be possible to specifically target the major muscle groups affected by Duchenne muscular dystrophy (Gregory et al. 2004, Gene Therapy 11(14):1117-25). Highly efficient transfer of the β-galactosidase gene to these major muscle groups supported the potential for in utero gene delivery for therapeutic and long-term prevention or correction of muscular dystrophies. In utero gene delivery allowed the transfer of the human factor IX gene into the foetal circulation of immunocompetent haemophiliac mice resulting in permanent therapeutic correction of haemophilia B (Waddington et al. 2004, Blood 104:2714-2721). These studies demonstrated the potential of in utero gene delivery for targeted gene delivery and gene therapy.

According to the invention, somatotransgenic bioimaging is a non-invasive technique allowing tissues to be specifically targeted without requiring animal sacrifice or solely relying on peripheral, secreted or excreted body fluids or the taking of tissue biopsies. Lentiviral constructs are generated with a bioluminescent reporter gene under the control of a genetic element of interest. The gene construct can be specifically targeted to a site or tissue of interest in a foetal or neonatal animal. Specific targeting is achieved by purposely delivering the vector to the site or tissue of interest in the foetal or neonatal animal, for example by injection. An additional layer of specificity may be provided by the use of lentiviruses that are pseudotyped with envelopes that increase the tissue-specificity of gene transfer. Specific targeting of the bioluminescent reporter gene to a site or tissue of interest reduces the significant and complex background interference during imaging which could otherwise occur using standard germline transformed transgenic animals due to the expression of the reporter gene in all cells. This is because the transgene is only expressed in the tissues to which it has been delivered by the vector, so the observed bioluminescence comes only from those tissues, not from all tissues. In other words, because the vector is delivered to specific tissues, the effect of a pathology or therapy on those tissues in particular can be studied more precisely and reliably. A somatotransgenic approach would also provide continual readout throughout application of a drug or metabolite.

Once the bioluminescent reporter gene under the control of the genetic element of interest has been targeted to the required site or tissue in the foetal animal, the animal is allowed to mature to term and adulthood. In the case of studies evaluating drug metabolism or toxicity, the primary tissue to be targeted is the liver. It is then possible to monitor the expression of the bioluminescent reporter gene in the animal in response to controlled events. The invention makes it possible to carry out whole-animal bioimaging, preventing the need for animal sacrifice, complex surgery or the need to rely on peripheral, secreted or excreted body fluids. The use of lentiviruses in particular results in efficient, integrative gene transfer and stable gene expression throughout the life of the animal allowing bio-imaging to be performed at any life-stage of the animal. Furthermore, prenatal gene transfer results in animals immune-tolerised to the transgenic material.

Moreover, the technique of the invention is quicker than conventional, whole-body transgenesis because all it requires is to make a vector and deliver it to the appropriate site in the foetal or neonatal animal, then allow the animal to develop in the normal way. In conventional transgenesis, it is of course necessary to carry out the transformation at a much earlier stage.

Also, in conventional transgenesis, all the cells of the transgenic animal ultimately arise from the same transformation event in the same cell, i.e. the transgene is in the same place and orientation in the genome of every cell. In the present invention, the transduction is carried out at the tissue level (somatotransgenesis) so there will be many different individual transformation events in many different individual cells. This means that position effects are avoided. In a conventional germline transgenic, if the vector integrates in an unfavourable location, that unfavourable result will exist in all the animal's cells and may give a misleading impression in any analysis. In a somatotransgenic animal according to the invention, any unfavourably positioned insertions will be compensated for by other, favourably positioned ones.

A further advantage is that, according to the invention, non-integrating vectors may be used where appropriate, whereas in a conventional transgenic an integration event would always be required, otherwise the transgene would not be replicated into every cell of the resulting animal

Another advantage of somatotransgenesis is that the luciferase can be introduced into any transgenic or knockout mouse model or background strain. In contrast, conventional germline transgenics have to be crossed onto these different strains, and achieving genetic homogeneity by the fastest method, speed congenics, still takes at least 10 generations.

According to the invention it is possible to monitor the progression of a pathology in a model animal using the technique of somatotransgenic bioimaging. The background of the animal upon which in utero gene delivery is carried out can be varied and used to determine which pathology is being modelled. An advantage of the non-invasive nature of bioimaging is that the expression of the reporter gene and progression of the pathology can be continually or consecutively monitored. Expression can for example be monitored before, during, after or throughout pathology-defined events. Bioluminescence can be monitored before and after the administration of a compound to determine the effect of the compound on the expression of the reporter gene. The invention is therefore useful for determining the efficacy of candidate therapeutic compounds. The effect of compounds on an animal model can be analysed in detail through the ability of the technique to provide a continual bioluminescence read-out. The technique of the invention is advantageous because this can be carried out in the context of known and proven models, in such a way that the effect of a pathology or therapy on particular tissues can be studied.

A non-invasive model of endometriosis for monitoring the efficacy of antiangiogenic therapy was provided in Becker et al. 2006 (Am. J. Path., 168:2074-2084). Germline integrated luciferase-expressing transgenic mice were generated with the luciferase gene under the control of the human ubiquitin C promoter. The mice demonstrated full-body bioluminescence. Endometrial tissue from these transgenic mice was surgically removed and implanted into nonluminescent recipients. The model provided a means of imaging endometriotic lesions, monitoring endometriotic growth and the efficiency of antiangiogenic therapy in the treatment of endometriosis. This model differs significantly from the current invention. The current invention enables a wide variety of tissues to be targeted individually and investigated non-invasively without the need for surgery.

The invention therefore provides: a method for determining whether the expression of a reporter gene is modulated by a compound, said method comprising: (a) administering said compound to a non-human transgenic animal, generated by gene transduction of one or more specific tissues when in utero or neonatal, with a vector comprising a bioluminescent reporter gene operably linked to a genetic element responsive to a pathology or therapy; and (b) determining the effect, if any, of said compound on the expression of said reporter gene in said specific tissue or tissues, said determination comprising detecting from the animal bioluminescence caused by the activity of the gene product of the reporter gene.

The invention also provides the use of a non-human transgenic animal generated by gene transduction of one or more specific tissues when in utero or neonatal, with a vector comprising a bioluminescent reporter gene operably linked to a genetic element responsive to a disease or therapy, for determining whether a compound modulates the expression of said reporter gene, by determining the effect, if any, of said compound on the expression of said reporter gene in said specific tissue or tissues, said determination comprising detecting from the animal bioluminescence caused by the activity of the gene product of the reporter gene.

According to the invention it is possible to monitor drug metabolism or toxicity in a wild-type animal model, a surgically or chemically induced disease model, a transgenic animal or a humanised animal model using the technique of somatotransgenic bioimaging. The background of the animal upon which in utero gene delivery is carried out can be varied to assay drug metabolism in a disease state that could be different from the steady state. An example would be the metabolism of chemotherapy drugs in animals with advanced hepatocellular carcinoma. An advantage of the non-invasive nature of bioimaging is that the expression of the reporter gene can be continually or consecutively monitored. An alternative strategy is to use “humanised” mouse models that have partial or complete ablation of the host's hepatocytes concomitant with reconstitution with human hepatocytes. Such protocols have been described using different technologies by Katoh et al. 2007 (Katoh, M. et al. 3 Pharm Sci; 96: 2, p428); Turrini et al. 2006 (Turrini, P. Transplant Proc; 38: 4 p1181) and Mitchell et al. 2002 (Mitchell, C. Am J Pathol; 160:1 p31). A differential in the responsiveness of human and mouse CYPs would suggest that such a humanised mouse model would be invaluable in assaying relative expression of human CYPs in an animal model. Our strategy would be to genetically manipulate human fetal, neonatal or adult hepatocytes with CYP-specific reporter constructs ex vivo using viral vectors. The genetically modified hepatocytes would then be used to reconstitute a murine liver niche and the resultant animal used in somatotransgenic bioimaging assays to assess drug metabolism and/or effect.

The plasticity of our model means that any CYP for which there is defined promoter-enhancer sequence can be utilised on a human or non-human background, in a disease model or toxicity model and data is generated over the complete time of the experiment avoiding potentially both species and individual variations.

The invention therefore provides a method of evaluating the metabolism and/or toxicity of a compound comprising:
(a) administering said compound to a non-human transgenic animal, generated by gene transduction of one or more specific tissues when in utero or neonatal, with a vector comprising a bioluminescent reporter gene operably linked to a genetic element responsive to drug metabolism and/or drug toxicity; and
(b) determining the effect, if any, of said compound on the expression of said reporter gene in said specific tissue or tissues, said determination comprising detecting from the animal bioluminescence caused by the activity of the gene product of the reporter gene.

The invention also provides a method of evaluating the metabolism and/or toxicity of a compound comprising:
(a) administering said compound to a non-human transgenic animal, generated by introduction, when in utero or neonatal, of transgenic cells comprising a bioluminescent reporter gene operably linked to a genetic element responsive to drug metabolism and/or drug toxicity; and
(b) determining the effect, if any, of said compound on the expression of said reporter gene in said introduced cells or cells derived from them, said determination comprising detecting from the animal bioluminescence caused by the activity of the gene product of the reporter gene.

The invention also provides the use of a non-human transgenic animal generated by gene transduction of one or more specific tissues when in utero or neonatal, with a vector comprising a bioluminescent reporter gene operably linked to a genetic element responsive to drug metabolism and/or drug toxicity, for determining whether a compound modulates the expression of said reporter gene, by determining the effect, if any, of said compound on the expression of said reporter gene in said specific tissue or tissues, said determination comprising detecting from the animal bioluminescence caused by the activity of the gene product of the reporter gene.

The invention also provides the use of a non-human transgenic animal generated by introduction, when in utero or neonatal, of transgenic cells comprising a bioluminescent reporter gene operably linked to a genetic element responsive to drug metabolism and/or drug toxicity, for determining whether a compound modulates the expression of said reporter gene, by determining the effect, if any, of said compound on the expression of said reporter gene in said specific tissue or tissues, said determination comprising detecting from the animal bioluminescence caused by the activity of the gene product of the reporter gene.

### BRIEF DESCRIPTION OF THE DRAWINGS

FIG. 1: demonstrates the differences between conventional analysis, conventional transgenic bioimaging and somatotransgenic bioimaging of the invention. In all three cases, the upper shaded region denotes the point at which disease induction takes place and the lower one denotes when a therapeutic molecule or candidate therapeutic molecule is introduced.

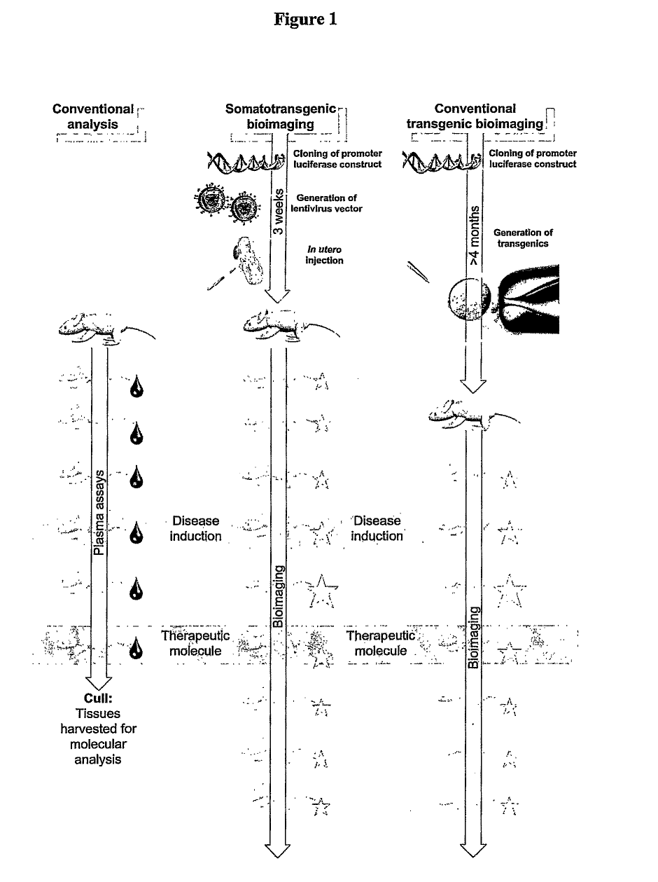

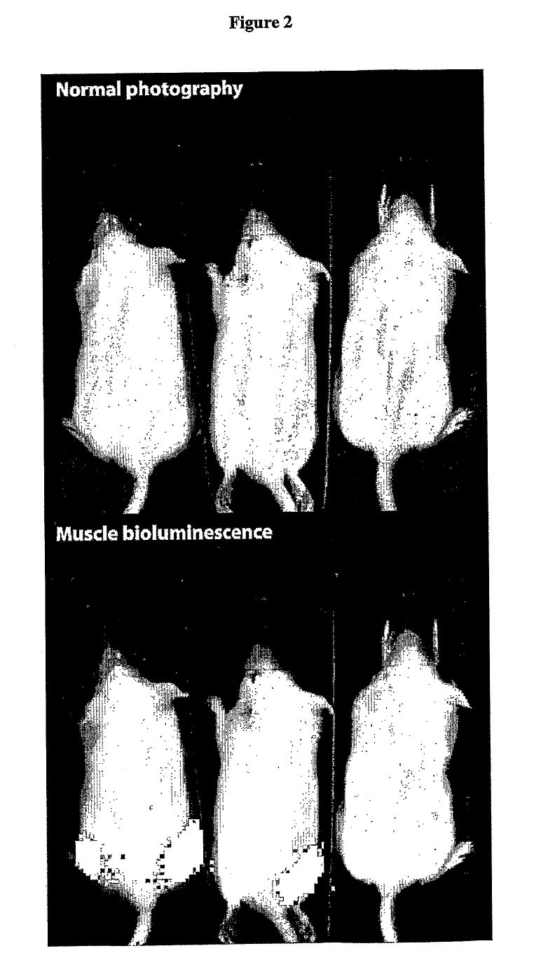

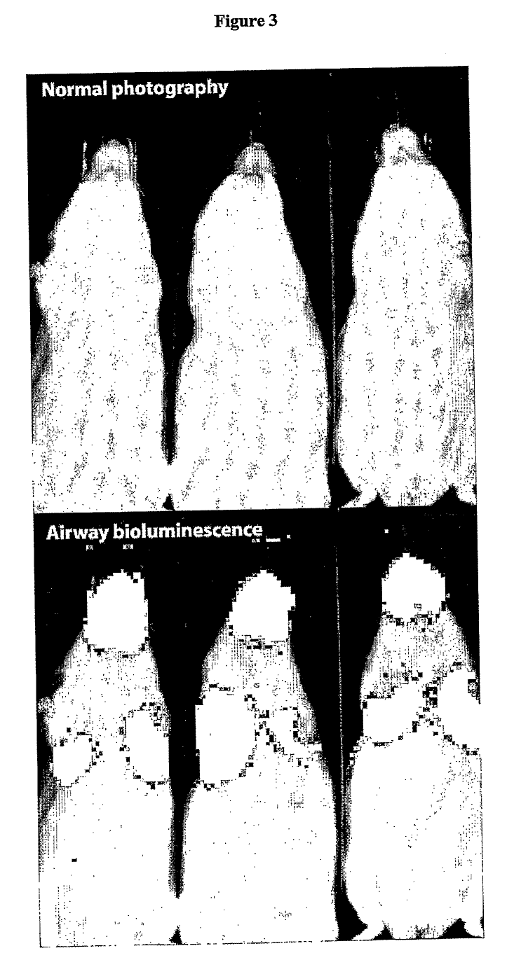

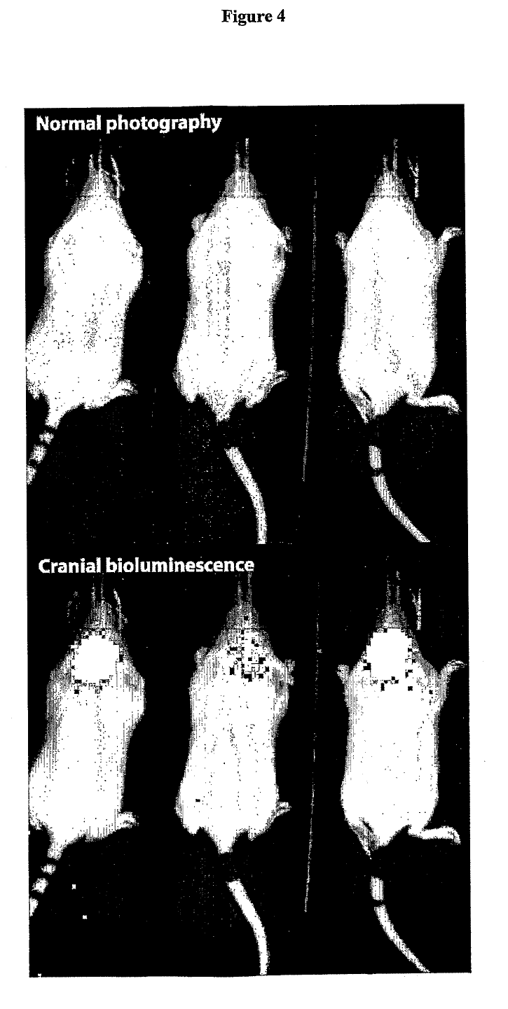

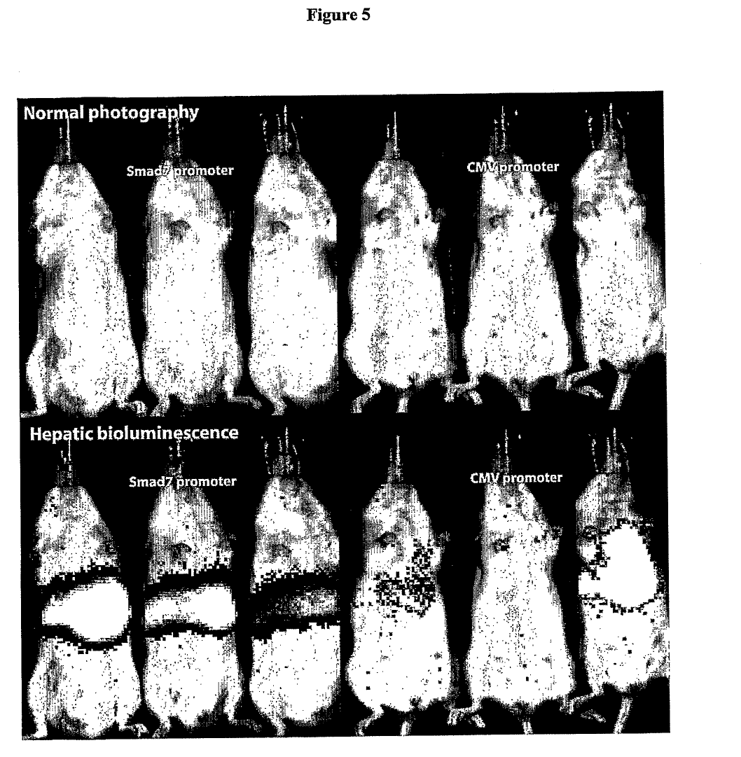

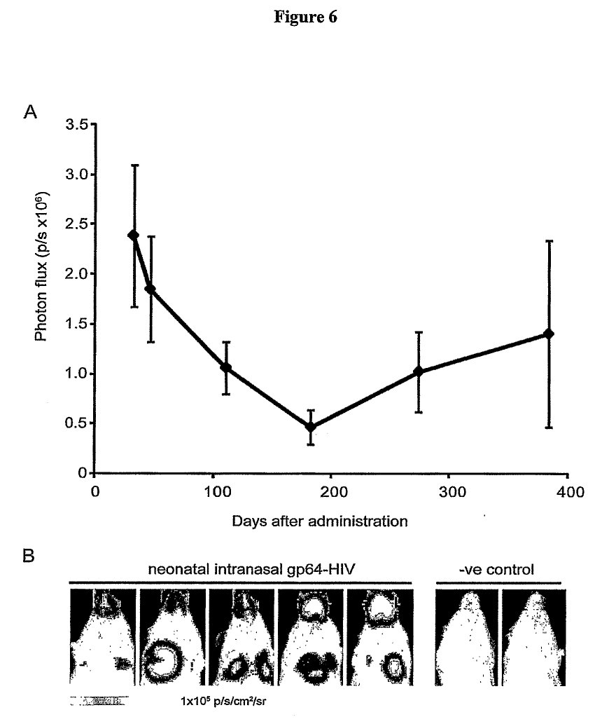

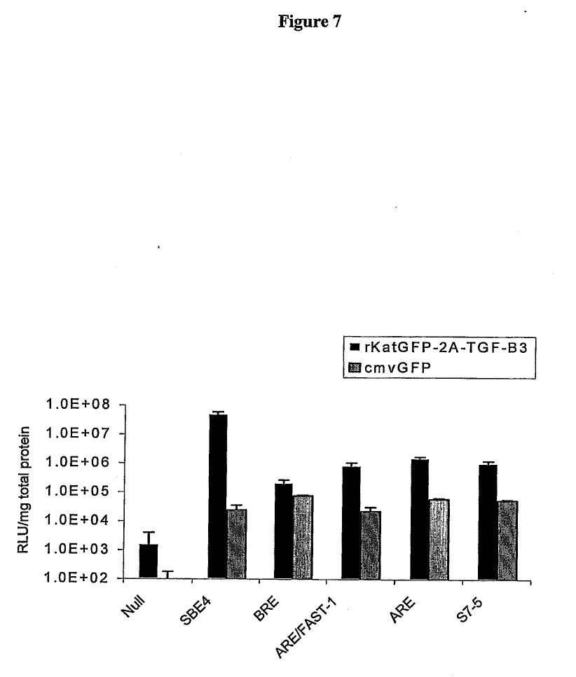

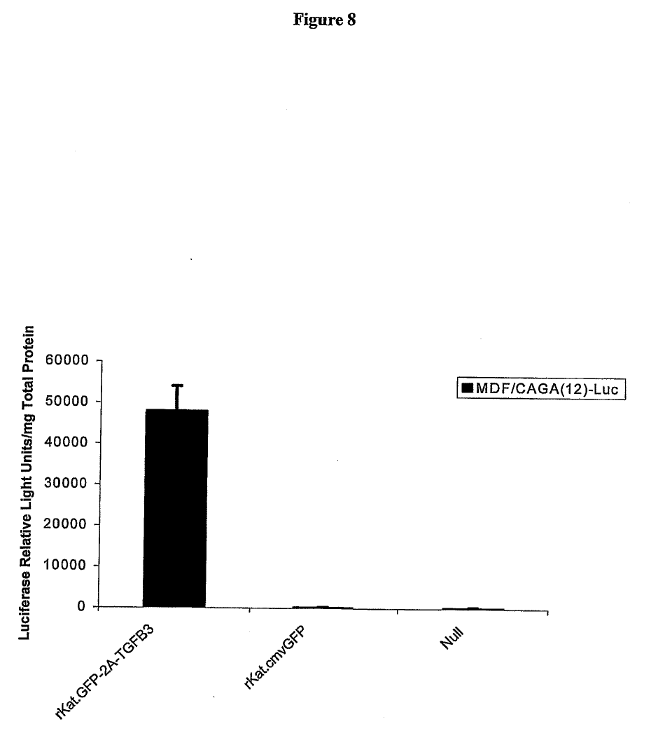

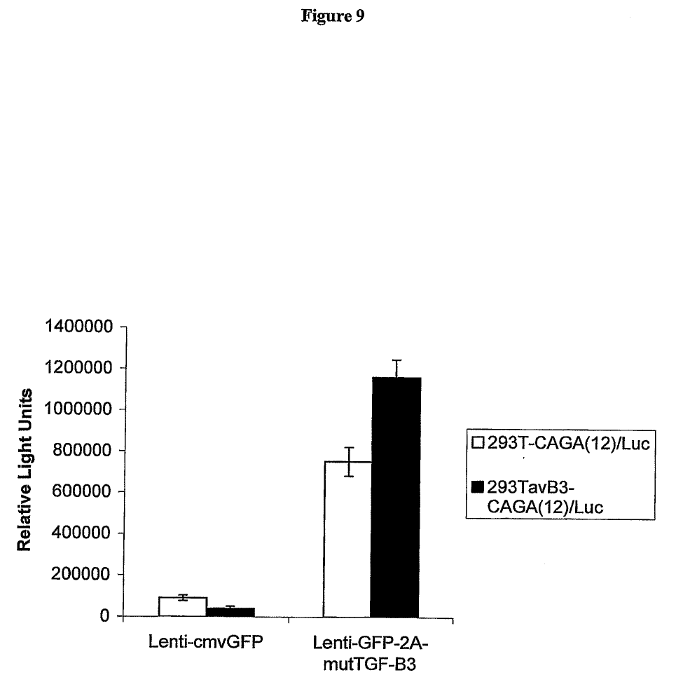

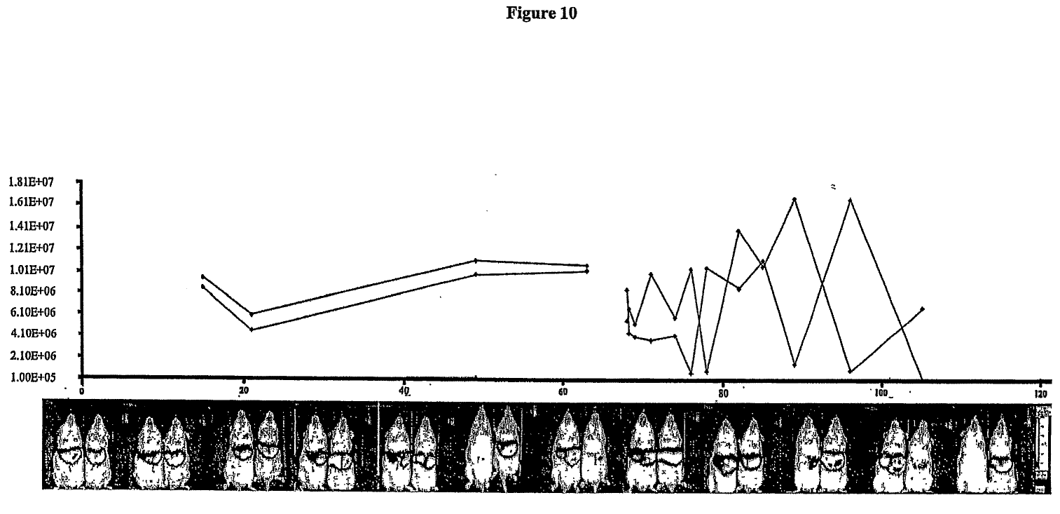

Conventional non-imaging analysis is shown on the left: plasma assays are carried out and the animal is ultimately culled so that its tissues can be harvested for molecular analysis.

Conventional (germline) transgenic bioimaging is shown on the right. At the top, cloning of a promoter luciferase construct is shown, followed below by generation of transgenics, lasting over four months. Bioimaging is then carried out.

An illustrative embodiment of somatotransgenic bioimaging according to the invention is shown in the centre. Compared to conventional transgenic bioimaging, the step of generation of transgenics lasting over four months is replaced with generation of a lentiviral vector and in utero injection of this vector. This takes about three weeks.

FIG. 2: Muscle bioluminescence following neonatal intramuscular injection of lentivirus vector where luciferase is driven by a constitutive promoter. Normal photography in upper panel, muscle bioluminescence in lower panel.

FIG. 3: Airway bioluminescence following neonatal airway instillation of lentivirus vector where luciferase is driven by a constitutive promoter. Normal photography upper panel, airway bioluminescence in lower panel.

FIG. 4: Cranial bioluminescence following fetal intracranial injection of lentivirus vector where luciferase is driven by a constitutive promoter. Normal photography upper panel, airway bioluminescence in lower panel.

FIG. 5: Hepatic bioluminescence following neonatal intravascular injection of lentivirus vector where luciferase is driven by a TGF-beta-sensing promoter. Normal photography in upper panel, hepatic bioluminescence in lower panel.

FIG. 6: demonstrates long-term transgene expression in the lung (airway) following neonatal airway instillation of lentivirus vector where luciferase is driven by a constitutive promoter. A single dose intra-amniotic administration of gp64/HIV-luciferase (˜3×10⁷ iu) was applied to day 1 neonatal mice (n=5). These animals, along with uninjected controls (n=2), were imaged after intra-nasal administration of 50 μl of 15 mg/ml luciferin.

Luciferase expression in the lungs is shown after removal of background (control) values and is detectable for the length of the study (390 days) (A). Graphic representation of luciferase expression in the lungs and noses of the above mice (B). Images were taken 384 days of age (B). Scale bars represent 100 μm.

FIG. 7: NIH-3T3 cells were transfected with plasmids containing TGF-β3 responsive elements driving luciferase expression. These cells were then transduced with a retroviral vector expressing TGF-433. The SBE4 responsive element is specific to TGF-β activation via smad2/3 mediated transcriptional activation. This Smad activation can be further delineated to Smad2 specific transcriptional activation using the ARE responsive element in conjunction with the xenopus Fast-1 transactivator (ARE alone is only Smad2/3 specific). The BMP-specific responsive element activates through Smad1/5/8 activation and should not be responsive to TGF-β3 activity. Finally, Smad7 is an inhibitor Smad and is known to be upregulated in a negative feedback loop by TGF-β3 activation. Transgenic TGF-β3 activation upregulates the SBE4 element by ˜1000-fold over controls and the ARE, ARE/Fast-1 responsive elements and Smad7 promoter all show significant responses over controls. The negative control BMP responsive element BRE did not show a significant response over controls when subjected to TGF-β3 over-expression. We conclude that in vitro, these responsive elements are reactive to TGF-β3 activation.

FIG. 8: A cell line transgenic for a synthetic TGF-13 responsive element driving the firefly luciferase gene was generated from primary mouse dermal fibroblasts. The CAGA(12) Smad Binding Element (SBE) is placed upstream of a minimal promoter and will respond to Smad2/3 specific transcriptional activation. Primary murine dermal fibroblasts (MDF) were transduced with a lentiviral vector containing the CAGA(12)-Luc element. These cells were then incubated in conditioned medium from MDFs transduced with a lentivector expressing either TGF-β3 or GFP. The MDF-CAGA(12)-Luc cells showed significant luciferase response to conditioned medium from TGF-133 over expressing cells compared to control. These data confirm that we are able to generate transgenic cells responsive to TGF-β activity from primary murine cells.

FIG. 9: Human embryonic kidney 293T cells stably expressing the human αvβ3 integrins and control 293T cells were transduced with the Lenti/CAGA(12)-Luc vector to generate two transgenic lines. Again, these cells were subjected to conditioned medium from cells either over-expressing TGF-β3 or control cells. Luciferase output was significantly enhanced in the αvβ3 expressing cell lines compared to the control 293T cells. We can conclude that the expression of αvβ3 integrins enhances TGF-β3 responsivity in 293T cells.

FIG. 10: Hepatic bioluminescence following neonatal intravascular injection of lentivirus vector where luciferase is driven by a TGF-beta-sensing promoter. Quantitation of bioluminescence upper panel. Standardised bioluminescence images lower panel. Foetal mice (E17) were injected via the intravascular route with a VSV-G-pseudotyped HIV luciferase vector. The luciferase transgene was driven by the TGF-β1 activated, Smad-specific response element CAGA(12). Resultant somatotransgenic progeny were assayed at four times over 60 days before being subject to bile duct ligation, an accepted method of inducing liver injury and fibrosis. Mice were continually assayed as liver fibrosis progressed. Assay of luciferase expression consisted of photography of the anaesthetised mice using a CCD camera five minutes after intraperitoneal injection of luciferin.

### DETAILED DESCRIPTION OF THE INVENTION

### Vectors

Preferred vectors of the invention are viral vectors. Viral vectors that can be used according to the invention include adenoviral, lentiviral, adeno-associated viral (AAV) and retroviral vectors or herpes simplex virus vector. Lentiviral vectors are preferred in many situations.

Integrating vectors, especially integrating lentiviral vectors, are preferred for many tissues, notably liver and lung. Non-integrating vectors, including integration-defective lentiviral vectors, may also be used in appropriate circumstances. Non-integrating vectors, for example AAV vectors, will find particular application in non-dividing tissues such as muscle and brain.

According to the invention, the vector comprises one or more bioluminescent reporter genes operably linked to one or more genetic elements responsive to a pathology or therapy.

In an alternative embodiment, the vector comprises a bioluminescent reporter gene operably linked to a genetic element responsive to drug metabolism and/or drug toxicity.

Preferred bioluminescent reporter genes are luciferase genes. As is well known, the activity of luciferase on its substrate luciferin results in bioluminescence. Examples of luciferase genes that can be used according to the invention are the firefly luciferase gene, the activity of whose gene product on luciferin results in the emission of red (600 nm wavelength) light that penetrates body tissue and can thus be detected; and the sea pansy (renilla reniformis) luciferase gene, the activity of whose gene product on renilla-luciferin (coelenterazine) results in the emission of blue (466 nm wavelength) light for detection.

Typically, the vector cassette will contain an in vivo optimised luciferase gene with an upstream multicloning site where regulatory elements can be cloned in. Such regulatory elements would include enhancer and promoter elements from genes activated or repressed due to pathology progression or drug metabolism. Expression can be restricted using non-promoter genetic elements such as microRNAs.

Typically, for studying drug metabolism or toxicity, the promoter is a cytochrome P450 (CYP450) promoter or the promoter of a gene associated with cytochrome P450 activity. The promoter of any CYP450 gene involved in drug metabolism can be used, for example, a promoter from a human CYP450 or a CYP450 from the same species as the transgenic animal on which the testing is being conducted. Thus, in transgenic mice, it is preferred to use murine or human promoters. For example, a promoter from any of CYP1A2, 2A6, 2B6, 2C8, 2C9, 2C19, 2D6, 2E1 or 3A4 can be used. CYP3A4 promoters, particularly human and murine CYP3A4 promoters are preferred.

### Tissues

According to the invention, the vector is introduced into one or more specific tissues, leaving others unaffected (or at least much less affected, such that they can be considered in practice to be non-transgenic). This differs from the position in conventional bioimaging where the transgenic animal is a germline transgenic that carries the transgene in all cells of all tissues. The introduction of the vector into one or more specific tissues has powerful advantages as discussed herein.

Preferred tissues into which to specifically introduce the vectors of the invention include liver, heart, kidney, muscle, brain, thyroid, lung, pancreas, blood, spleen, thymus, testis, gut (e.g. oesophagus), trachea, vascular system, peripheral nervous system and eye tissues. Lung and liver are especially preferred.

Various mechanisms can be used to target the vector specifically to particular tissues, as discussed herein.

### Animals

It is preferred to apply the techniques of the invention to (non-human) mammals. Rodents such as rats, mice and rabbits, and primates, such as monkeys, are preferred. Mice are particularly preferred, because of the large body of knowledge concerning transgenic mice, including the availability of a full-genome sequence, and the wide availability of established mouse disease models, and for experimental convenience. There is also the existence of a number of mouse models with humanized livers thereby presenting human tissue in an in vivo context. Mini-pigs or small primates can also be used. In general, smaller animals are preferred to larger animals because it will be easier to detect bioluminescence coming from a tissue within a smaller animal. Different techniques may need to be applied to deliver vectors optimally to different animals.

### Tissue Specificity

In general, vector delivery to the foetal or neonatal animal will be by injection, either into the tissue concerned or systemically.

In mice, systemic injection has been shown to direct lentiviral vectors specifically to spleen, liver, lungs and heart. Such targeting can be achieved via intravascular injection into the vessels of the foetal yolk sac; or into the superficial temporal vein of neonatal animals. A further level of targeting is achieved by utilising a variety of tissue tropic viral envelope glycoproteins with which to pseudotype the lentiviral vector.

An attraction of lentivirus vectors, which is shared by some other vectors, is the potential for use of different surface receptors to alter the tissue and cell tropism of the vector, a process known as pseudotyping. Whereas lentivirus vectors may be synthesised to contain the surface glycoproteins from other viruses (explained in more detail below), adeno-associated virus vectors and adenovirus vectors can be derived with envelope proteins from other serotypes from the same genus to confer different tropisms. For example, AAV serotype 9 confers much stronger tropism to cardiac cells than AAV serotype 8 which has a greater tropism to liver cells. Different adenovirus serotypes can therefore be used. Different adenovirus serotypes possessing fibres of other serotypes can also be used.

Lentivirus vectors are produced by transfecting cells with three or four plasmids containing separate components of DNA to produce a virus-like vector particle. Three plasmid systems include the packaging plasmid consisting of essential viral components including the gag and pol genes for synthesis of a virus particle. The second plasmid contains the “payload” such as luciferase cDNA driven by a chosen promoter flanked by terminal repeats. This also contains a packaging sequence which ensures that the payload is incorporated into the virus particle. The third plasmid encodes the glycoproteins which coat the envelope and confer the vector with tropism for specific cell types. Many different and divergent viral envelopes have been described for pseudotyping retrovirus vectors (lentiviruses are a genus of the retrovirus family; HIV is a subgenus). These pseudotypes include G protein of vesicular stomatitis virus (VSV-G), and glycoproteins from influenza, parainfluenza, ebola, gibbon ape leukaemia virus, lymphocytic choriomeningitis virus (LCMV) and baculovirus (gp64) amongst others.

We and others have observed specificity for certain tissues or organs depending upon the pseudotype which coats the virus and depending upon the route of administration to the young organism. For example, VSV-G imparts tropism for the animal's liver and spleen after intravenous injection. gp64 imparts a tropism of airway epithelia after intra-amniotic or intranasal delivery, whereas intranasal VSV-G hits has a tropism for alveolar cells. Rabies envelope glycoprotein provides a strong tropism for the peripheral nervous system and dorsal root ganglia after intravenous vector delivery. The targeting of hepatocytes can be achieved using appropriate pseudotypes such as Ebola, gp64, VSV-G and HA/HN.

Targeting can also be achieved by controlling the site of delivery at a physical level, i.e. by delivering the vector specifically to the tissues in which it is required. This can be applied instead of, or as well as, pseudotyping-based approaches. In mice at least, intramuscular injection will result in gene expression in the hind limb, although not specifically in muscle. Intrathoracic injection targets the respiratory musculature, notably the important diaphragm. Supracostal injection also targets the respiratory musculature. Intraperitoneal injection achieves expression in either the peritoneal mesothelium or abdominal muscles and diaphragm. Intra-amniotic injection can be used for lung and nasal targeting.

Intraspinal and intracranial injection target the peripheral or central nervous system. Intrahepatic injection targets the liver.

For some examples of tissue-specific delivery methodology that can be used according to the invention, see: S. N. Waddington et al. 2003, Gene Therapy 10:1234; Gregory et al. 2004, Gene Therapy 11(14):1117-25 (injection into foetal skeletal muscle of hind limb, systemic injection via foetal yolk sac vessels, intraperitoneal injection); and Waddington et al. 2004, Blood 104:2714-2721. For an example of liver tissue-specific delivery methodology that can be used according to the invention, see: Waddington et al. 2004, Blood 104:2714-2721.

### Somatotransgenesis

Typically, foetal or neonatal animals, preferably mice, are injected at specified developmental timepoints via a number of specified routes with a solution containing a vector of the invention, typically a viral vector, in order to achieve spatial and temporal tissue targeting. The infection provides a genome integrated or episomally persisting transgene that is immune tolerised and acts as a genetic effector in the desired tissue type of any experimental animal at birth. This process allows the investigator to choose both the readout and background. For example, it is possible to build on a base of a disease model transgenic or knockout mice adding surgically or chemically induced disease states as well as drug application and provide a clearly defined real-time readout of a specified downstream marker for a therapeutic.

In foetal mice, depending on the precise type of delivery/targeting required, the preferred time for injection within the 20-day gestation period is normally from 10 days post-conception (dpc) to birth, e.g. at 11, 12, 13, 14, 15, 16, 17, 18, 19 dpc. 12 to 17 dpc is preferred and 16 dpc particularly preferred for delivery to the liver. In neonatal mice, the preferred time will again depend on the precise type of delivery/targeting but will generally be from birth to 20 days post-birth, e.g. 10 to days post-birth, or from 1 to 5 days post-birth, and especially 1, 2 or 3 days post-birth. For other animals, equivalent time periods may be defined on a developmental basis.

### Validation of Drug Candidates

According to the invention, the activity of drug candidates against a wide variety of pathologies can be investigated. All that is required is an existing model animal, typically a mouse model; and a genetic element, typically a promoter or enhancer, that is responsive to the pathology and/or to a therapy for it. Many of both of these are available. Foetal or neonatal individuals of the model animal are subjected to somatotransgenesis by the techniques discussed herein, using vectors of the invention in which the pathology-responsive element is operably linked to a bioluminescent reporter gene. The tissue(s) for transformation is (are) chosen such that the vector is targeted to one or more tissues affected by the pathology in question. For example, if it is desired to validate drug candidates against a liver pathology, targeting would typically be to the liver.

When the somatotransgenic animals mature, they retain the model characteristics but also possess the pathology-responsive element/bioluminescent reporter transgene combination in one or more tissues that will be affected by the pathology when it is induced. Bioimaging may be carried out at this point to act as a control. Then, if necessary, the pathology is induced in the appropriate manner, e.g. chemically and/or by surgery. Bioimaging is then typically carried out to measure the bioluminescence caused by the activity of the pathology/therapy-responsive element and the expression of the reporter gene to which that activity leads under disease conditions. Then the candidate compound is administered to the animal and bioimaging is typically carried out again and the results are compared. If the candidate compound has had an effect on the pathology, this will be apparent from the comparison. Normally, bioimaging will be carried out both before and after administration of the candidate compound. Under some circumstances, e.g. where the situation under disease conditions is sufficiently well understood, it may not need to be carried out before administration, only afterwards.

The effect, if any, of the candidate compound on the pathology may be determined qualitatively or quantitatively. In some cases, it may be desired to determine simply whether or not there is an effect; in other cases, the extent of the effect may be measured.

In this way, the effect of a candidate compound on a pathology can be determined. The response of a pathology to an already-known therapy can also be investigated.

### Pathologies and Models

Pathologies that can be investigated in this way include pathologies of the liver, heart, kidney, muscle, brain, thyroid, lung, pancreas, blood, spleen, thymus, testis, gut, trachea, vascular system, peripheral or central nervous system and eye. Any pathological pathway can be investigated. Pathologies of the lung or the liver, or of muscle and/or the nervous system are preferred.

Preferably, the lung pathology is selected from respiratory infections, asthma and chronic obstructive pulmonary disease (COPD). The respiratory infection may, for example, be caused by Respiratory Syncytial Virus (RSV), Parainfluenza virus (PIV) or Influenza Virus (IV). Preferably, the liver pathology is selected from liver fibrosis, liver cirrhosis and hepatitis C infection. Preferably, the pathology of the muscle and/or the nervous system is a degenerative disease, e.g. a disease selected from Duchenne Muscular Dystrophy (DMD), Myotonic Dystrophy (MD), Motor Neuron Disease (MND), Alzheimer's Disease (AD) and Huntingdon's Disease (HD).

An example of a disease model that can be investigated according to the invention is liver fibrosis. A liver fibrosis mouse model can be generated by fetal intravascular injection of lentiviral preparations. The lentiviral preparations comprise one or more genetic effectors involved in liver fibrosis upstream of a luciferase reporter gene, such as the ColIα2 promoter, Smad 7 promoter or BRE enhancer element. Continual bioimaging may be carried out before and after disease induction by a progressive fibrotic stimulus e.g. bile duct ligation, or by a chronic fibrotic stimulus e.g. CCl₄ administration. The effect of test compounds on the liver fibrosis model can be determined based on the bioluminescence read-out.

The effect of candidate anti-depressant drugs, such as Fluoxetine (Prozac), on a mouse model could be investigated using a lentiviral vector expressing the luciferase reporter gene under the control of genetic elements, for example those that regulate the expression of the 5-HT transporter and/or receptor. The lentiviral preparation may be applied by fetal intracranial injection. The effect of candidate compounds may be determined by continued bioimaging before, during and after administration of the candidate anti-depressant drug.

The invention can also be applied to situations in which the pathology is inflammation, or in which the pathology comprises or gives rise to inflammation, for example in liver, lung or joints but potentially also elsewhere. To evaluate the effect, if any, on inflammation of a candidate compound, somatotransgenesis is carried out as discussed, in a model animal that has, or can be made to have, inflammation in e.g. liver, lung, joints, muscle, heart, brain or other organs with the vector containing a genetic element responsive to inflammation and/or to the relief of inflammation; for example a specific promoter that is upregulated due to inflammation. The vector will be delivered to suitable tissues in which signals from the inflammation will cause the responsive element to be activated and express the bioluminescent gene such that bioimaging can be carried out. Then the effect of a candidate compound can be evaluated, qualitatively and/or quantitatively, by carrying out bioimaging, normally both before and after administration of the compound, but (see above) possibly only afterwards under some circumstances.

According to the invention, a mouse model of inflammation may be generated using a lentiviral vector expressing the luciferase reporter gene under the control of NFkB enhancer elements and a murine minimal promoter. The lentiviral preparation may be applied by fetal intra-amniotic injection. Anti-inflammatory drug effects may be modeled in the mature mouse by continued bioimaging before, during and after administration of anti-inflammatory drugs.

### Evaluation of Drug Metabolism & Toxicity

Foetal or neonatal animals are subjected to somatotransgenesis by the techniques discussed herein, using vectors of the invention in which a genetic element responsive to drug metabolism and/or drug toxicity is operably linked to a bioluminescent reporter gene. The tissue(s) is (are) chosen such that the vector is targeted to one or more tissues that are affected by drug metabolism and/or toxicity. The tissue of greatest interest is thus normally the liver since that is the primary site of drug metabolism and detoxification, and the main site of expression of CYP450 genes from which the preferred promoters of the invention are derived.

When the somatotransgenic animals mature, they possess the responsive element/bioluminescent reporter transgene combination in one or more relevant tissues. Bioimaging may be carried out at this point to act as a control. Then, the candidate compound is administered. Bioimaging is then typically carried out to measure the bioluminescence caused by the activity of the responsive element and the expression of the reporter gene to which that activity leads when the responsive element is active. If the candidate compound has been metabolised or shown toxicity, a change in the activity of the responsive element will be observed. Normally, bioimaging will be carried out both before and after administration of the candidate compound. Under some circumstances, e.g. where the situation prior to administration is sufficiently well understood, it may not need to be carried out before administration, only afterwards.

The effect, if any, of the candidate compound may be determined qualitatively or quantitatively. In some cases, it may be desired to determine simply whether or not there is an effect; in other cases, the extent of the effect may be measured.

In preferred embodiments, a vector comprising a CYP450 promoter, or the promoter of a gene associated with CYP450 activity, operably linked to a bioluminescent reporter gene such as a luciferase gene, will be introduced somatotransgenically into the liver of a foetal or neonatal mouse, e.g. by systemic injection, and bioimaging will be carried out before and after administration of a candidate compound; and the comparison between the measurements thus obtained will be used to determine whether and/or to what extent the promoter has been activated by the candidate compound, i.e. whether and/or to what extent the compound has been metabolised in the liver and/or demonstrated toxicity.

### Use of Transgenic Cells

In some embodiments, the response element/reporter combination of the invention may not be introduced directly into the animal by means of a vector; rather, transgenic cells comprising the combination are introduced. In these embodiments, cells, for example cells of foetal, neonatal or adult origin are transduced and the cells are introduced into the animal, normally by injection as discussed above. Typically, this will be achieved with a viral vector of the invention, especially a lentiviral vector, as discussed above, although any suitable vector may be used.

Normally, the cells will be introduced into the tissue or organ from which they themselves originate.

In some embodiments, the cells will be cells of an animal of the same species as the animal into which they are introduced, e.g. mouse cells will generally be introduced into mice.

Alternatively, the cells may be cells of human origin (fetal, neonatal or adult) and be introduced into a compatible non-human animal, e.g. one that is “humanised” (see above). In this case of liver cells, this means that they may be introduced (normally injected) into a normal or partially tissue-ablated liver of either a wild-type experimental animal (commonly a mouse) or an immunosuppressed one. Repopulation of the host liver with human hepatocytes can be facilitated by either chemical or biological ablation of host cells or the allowance of the cells to repopulate in conducive conditions.

In preferred embodiments, the cells will be liver cells, particularly hepatocytes. Normally, these will be introduced into the liver of the animal, typically by injection.

### Bioimaging

To assess the results of the processes described above, such as the administration of a candidate compound, bioimaging is carried out according to known techniques. For example, the process of whole body imaging is described in a review by Contag, C. H., and Bachmann, M. H. (Advances in in vivo bioluminescence imaging of gene expression. Ann. Rev. Biomed. Eng. 4:235-260; 2002); and also in several papers by the same authors.

With luciferase, such bioimaging is non-invasive except for injection of the luciferin substrate on which the luciferase acts to produce bioluminescence. However, luciferin can also be administered non-invasively in drinking water so the imaging could be done with the animal conscious.

Bioluminescence can be detected in any suitable manner, e.g. using a charge coupled device (CCD) camera.

Typically, the animals will eventually be sacrificed once all required measurements have been taken.

The invention is illustrated by the following Examples

### EXAMPLES

### Example 1

Experiments were conducted to determine the possibility of achieving long-term tissue-specific transgene expression in mice.

### Vector Production and Validation

The gp64- and vsvg-pseudotyped luciferase vector used for long-term analysis was produced as previously described by Seppen et al (Seppen J, Rijnberg M, Cooreman M P, Oude Elferink R P. Lentiviral vectors for efficient transduction of isolated primary quiescent hepatocytes. J Hepatol 2002; 36: 459-465).

Lentivectors were prepared as follows: Producer 293T cells were seeded at 2×10⁷ cells per T-150 flask. The next day, plasmid DNA was mixed in the following amounts per T-150 flask; vector construct (pHR.SINcpptSEW) 40 μg, pMDG.2/pHCMVwhvGP64 10 μg, pCMVΔ8.74 30 μg to a final volume of 5 ml in OptiMEM (Invitrogen, Paisley, UK). Polyethylenimine (PEI, 25 kDa) (Sigma, Poole, UK) was added to 5 ml of OptiMEM to a final concentration of 2 μM and filtered through a 0.22 μm filter. The DNA was added dropwise to the PEI solution and incubated at room temperature for 20 minutes. The DNA/PEI solution was added to the 293T cells and incubated for 4 hours at 37° C., 5% CO₂ before being replaced by complete DMEM (Invitrogen). Supernatant was harvested after a further 48 h and replaced with growth medium for a second collection after 72 h if necessary.

Viral supernatant was initially centrifuged at 2500 rpm using a desktop centrifuge (MSE, Germany) for 10 minutes and then filtered through a 0.22 μm filter prior to ultracentrifugation (Sorvall, UK) at 23,000 rpm (˜100,000 xg), 4° C., for 2 h. Medium was carefully decanted and viral pellets resuspended in 300 μl of PBS medium. Finally, viral suspensions were centrifuged at 4,000 rpm for 10 minutes using a desktop microfuge to remove any remaining debris. All viral preparations were used fresh and titred by Reverse Transcriptase qPCR and p24 ELISA assay as previously described (Logan A C, Nightingale S J, Haas D L, Cho G J, Pepper K A, Kohn D B. Factors influencing the titer and infectivity of lentiviral vectors. Hum Gene Ther 2004; 15: 976-988).

### Animal Studies

Male and female MF1 mice (Harlan, UK) were used. For in utero administration, time-mated pregnant mice were anaesthetised by inhalation of isofluorane (Abbott Laboratories, UK). A midline laparotomy was performed and both horns of the gravid uterus exposed. All injections were performed by transuterine injection.

For fetal airway administration, each amniotic cavity was injected (50 μl volume) by penetration of the uterus wall, the yolk sac and amniotic membranes with a 33-gauge Hamilton Microliter Syringe™. For neonatal airway administration, 20 μl of vector was applied (2×10 μl doses) to the nostrils and the neonate inhaled the vector (results in FIG. 3, normal photography upper panel, airway bioluminescence in lower panel and FIG. 6, long-term lung bioluminescence, constitutive & ubiquitous promoter). For fetal intravascular injection, a 34-gauge needle (Hamilton, UK) was used to perform a transuterine injection of 20 μl solution into a peripheral yolk sac vessel. For neonatal intravascular injection, the neonate was subject to hypothermic anaesthesia and 40 μl injected into the superior temporal vein (results in FIG. 5, normal photography in upper panel, hepatic bioluminescence in lower panel and FIG. 10, bioimaging before and after bile duct ligation, TGF-beta-sensing promoter). For neonatal intramuscular injections 5 μl was injected directly into the leg muscle (results in FIG. 2—normal photography in upper panel, muscle bioluminescence in lower panel). For fetal intracranial injections, 5 μl was injected directly into the left hemisphere of the fetal mouse (results in FIG. 4—normal photography in upper panel, cranial bioluminescence in lower panel).

For all fetal injections, up to six fetuses were injected per dam. Following injection, the uterus was returned to the abdominal cavity and the abdominal wall closed in two layers with 5/0 Mersilk sutures (Ethicon, Brussels, Belgium). Animals were kept in a warmed cage in an undisturbed environment until awake and active. After neonatal administration, mice were allowed to recover on a thermostatically-warmed pad and returned to their mother. All animal work was carried out under United Kingdom Home Office regulations and was compliant with the guidelines of the Imperial College London ethical review committee.

### In Vivo Luciferase Bioimaging

Mice were anaesthetised with isofluorane (Abbott Laboratories, IL, USA) and 50 μl of 15 mg/ml D-luciferin (Gold Bio, MO, USA) was administered intra-nasally and imaged 5 mins later with a CCCD camera (IVIS, Xenogen, MA, USA), After acquiring a grey scale photograph, a 5 min bioluminescent image was obtained using 12 cm field of view, binning (resolution) factor of 8, 1/f stop and open filter. Regions of interest (ROIs) were defined manually (using a standard area in each case), signal intensities were calculated using the Living Image software (Xenogen) and expressed as photons per second. Background photon flux was defined from an ROI drawn over the control mice where no vector had been administered.

### Example 2

In order to assess long-teen transgene expression, a single intra-amniotic dose of gp64/HIV-luciferase (˜3×10⁷ iu) was administered to neonatal mice at day 1 (n=5). Mice were subjected to bioimaging over the course of one year and beyond and luciferase bioluminescence compared to controls (n=2). In vivo luciferase bioimaging was carried out as in Example 1. Luciferase expression was substantially above background throughout the analysis and persisted throughout this study (FIG. 6). The results demonstrate that significant expression is detectable up to one year after application.

### Example 3

Genetic bioeffectors useful in animal models were tested in vitro (Examples 3 to 5).

NIH-3T3 cells were transfected with plasmids containing TGF-β responsive elements driving luciferase expression. These cells were then transduced with a retroviral vector expressing TGF-β3. The SBE4 responsive element is specific to TGF-β activation via smad2/3 mediated transcriptional activation. This Smad activation can be further delineated to Smad2 specific transcriptional activation using the ARE responsive element in conjunction with the xenopus Fast-1 transactivator (ARE alone is only Smad2/3 specific). The BMP-specific responsive element activates through Smad1/5/8 activation and should not be responsive to TGF-β3 activity. Finally, Smad7 is an inhibitor Smad and is known to be upregulated in a negative feedback loop by TGF-β3 activation. The experiment was conducted as follows:

NIH-3T3 cells pre-plated at 1×10⁶ cells/well and transfected with 10 μg of reporter plasmid by standard Calcium Phosphate precipitation. After 48 hours cells were transduced with either rKat.TGF-β3 or a control rKat.cmvGFP retrovirus. The MLV-based retrovirus vector pKat/rKat system has previously been described (Finer, M. H., T. J. Dull, L. Qin, D. Farson, and M. R. Roberts. 1994. kat: a high-efficiency retroviral transduction system for primary human T lymphocytes. Blood 83:43-50). Retrovirus was prepared as follows: Producer 293T cells were seeded at 2×10⁷ cells per T-150 flask. Plasmid DNA was mixed in the following amounts per T-150 flask; vector construct 40 μg, pKat 10 μg, rKat to a final volume of 5 ml in OptiMEM (Invitrogen, Paisley, UK). Polyethylenimine (PEI) (Sigma-Aldrich, Poole, UK) was added to 5 ml of OptiMEM to a final concentration of 2 nM and filtered through a 0.2 μm filter. The DNA was added dropwise to the PEI solution and incubated at room temperature for 20 minutes. The DNA/PEI solution was added to the 293T cells and incubated for 4 hours at 37° C., 5% CO₂ before being replaced by complete DMEM (Invitrogen). Growth medium was changed after 24 h and supernatant harvested after a further 24 h and replaced with growth medium for a second collection if necessary. Viral supernatant was centrifuged at 5000×g for 10 minutes to remove cell debris and then filtered through a 0.22 μm filter. All viral preparations were used fresh and titered on 293T cells for biological titer by limiting dilution and FACS analysis for GFP. NIH-3T3 cells were transduced with the rKat retroviruses and 48 hours later conditioned medium was removed, filtered through a 0.45 μm nylon filter, and added to the plasmid containing NIH-3T3 cells. After 48 hours luciferase expression was measured in cell lysates. Cell lysate was assayed for luciferase expression using the Promega luciferase assay kit and a Berthold Flash'n'Glow LB955 (Berthold, Herts, UK) luminometer. Relative luciferase activity was expressed in arbitrary units with respect to total protein measured by standard Bradford assay as by manufacturer's instructions (BioRad, Herts, UK).

The results are shown in FIG. 7. Transgenic TGF-β3 activation upregulates the SBE4 element by ˜1000-fold over controls and the ARE, ARE/Fast-1 responsive elements and Smad7 promoter all show significant responses over controls. The negative control BMP responsive element BRE did not show a significant response over controls when subjected to TGF-β3 over-expression. We conclude that in vitro, these responsive elements are reactive to TGF-β3 activation.

### Example 4

A cell line transgenic for a synthetic TGF-β responsive element driving the firefly luciferase gene was generated from primary mouse dermal fibroblasts. The CAGA(12) Smad Binding Element (SBE) was placed upstream of a minimal promoter and will respond to Smad2/3 specific transcriptional activation. Primary murine dermal fibroblasts (MDF) were transduced with a lentiviral vector containing the CAGA(12)-Luc element. These cells were then incubated in conditioned medium from MDFs transduced with a lentivector expressing either TGF-β3 or GFP. The experiment was carried out as follows:

Murine dermal fibroblasts (MDF) were isolated as previously described by DiPersio et al. (DiPersio, C. M., S. Shah, and R. O. Hynes. 1995. alpha 3A beta 1 integrin localizes to focal contacts in response to diverse extracellular matrix proteins. J Cell Sci 108 (Pt 6):2321-36) and expanded to ˜60% confluence and transduced with a lentivector expressing a luciferase reporter gene under the control of either a smad 2/3-specific CAGA₍₁₂₎ promoter. Lentiviral preps were generated as described in Example 1. Cells were re-plated 48 hours later in the absence of serum and either subjected to rKat-TGF-β3 retroviral vector or a control cmvGFP vector and lysed 48 hours later. Cell lysate was assayed for luciferase expression using the Promega luciferase assay kit and a Berthold Flash'n'Glow LB955 (Berthold, Herts, UK) luminometer. Relative luciferase activity was expressed in arbitrary units with respect to total protein measured by standard Bradford assay as by manufacturer's instructions (BioRad, Herts, UK).

The results are shown in FIG. 8. The MDF-CAGA(12)-Luc cells showed significant luciferase response to conditioned medium from TGF-β3 over expressing cells compared to control. These data confirm that we are able to generate transgenic cells responsive to TGF-β activity from primary murine cells.

### Example 5

Human embryonic kidney 293T cells stably expressing the human αvβ3 integrins and control 293T cells were transduced with the Lenti/CAGA(12)-Luc vector to generate two transgenic lines. Again, these cells were subjected to conditioned medium from cells either over-expressing TGF-β3 or control cells. In more detail:

Human 293T cells stably transfected with αᵥ and β₃ integrin (293Tab) along with 293T controls were a kind gift from Dr. John Olsen, UNC, USA. 293T cells and 293Tabs were transduced with Lenti-CAGA₍₁₂₎-Luc and human DFs were tranduced with lentivirus either expressing human TGF-β3iresGFP, mutant TGF-β3iresGFP or a GFP control. Cells were cultured for a further 48 hours prior to trypsinisation and mixing of 293 cells and transduced DFs in a 1:1 ratio. Cells were incubated for a further 24 hours in serum containing medium then incubated for 48 hours in serum depleted medium supplemented with ITS+1 (Sigma-Aldrich). Cells were subsequently either FACS analysed, to assess the ratio of 293T cells to DFs, or lysed for the purpose of quantifying the luciferase expression.

The results are shown in FIG. 9. Luciferase output was significantly enhanced in the αvβ3 expressing cell lines compared to the control 293T cells. We can conclude that the expression of αvβ3 integrins enhances TGF-β3 responsivity in 293T cells.

### Example 6

Initial validation of the use of the novel somatotransgenic bioimaging technique for modelling pathologies in vivo has been using a mouse model of liver fibrosis. TGF-β specific profibrotic signalling is mediated through Smad signalling. We have chosen minimal enhancer/promoter elements defined in the literature and specific to such pathways to model the molecular consequences of portal liver fibrosis due to permanent occlusion of the common bile duct. This model of fibrosis parallels liver pathology as seen in biliary disease and Cystic Fibrosis (CF) liver disease.

With reference to FIG. 10, foetal mice (E17) were injected via the intravascular route with a VSV-G-pseudotyped HIV luciferase vector. The luciferase transgene was driven by the TGF-β1 activated, Smad-specific response element CAGA(12). Resultant somatotransgenic progeny were assayed at four times over 60 days before being subject to bile duct ligation, an accepted method of inducing liver injury and fibrosis. Mice were continually assayed as liver fibrosis progressed. Assay of luciferase expression consisted of photography of the anaesthetised mice using a CCD camera five minutes after intraperitoneal injection of luciferin. The novel technology has shown for the first time in vivo that Smad 2/3 signalling responds in a pulsing manner. This effect has most likely been missed previously when it has not been possible to continually monitor individual animals. Furthermore, even though both animals were treated identically they are not in synchrony. When averaged, this would nullify any different effect (FIG. 10).

### Example 7

Similar disease models can be created for Liver Cirrhosis and Hepatitis C infection as well as Pulmonary Fibrosis (PF). The sequencing of the human and mouse genomes has permitted the characterisation and availability of a wealth of highly specific DNA effector and repressor elements that can be incorporated into reporter cassette. It is equally feasible to use enhancer/promoter elements to assay muscular or nerve regeneration/degeneration or neuronal demyelination in order to model neuronal or muscle degenerating diseases such as Multiple Sclerosis (MS), Myotonic Dystrophy (MD), Muscular Dystrophies (DMD/BMD), Motor Neuron Disease (MND), Alzheimer's Disease (AD) and Huntingdon's Disease (HD). Furthermore, we are able to use pseudotyped lentiviruses to target the vascular endothelium facilitating the in vivo analysis of angiogenesis.

### Example 8

Lung pathology in Cystic Fibrosis (CF) has recently been addressed using small molecule drugs to knock down expression of or reduce the activity the n-subunit of ENaC, an epithelial sodium channel in the lung. An accurate model of CF lung disease has been developed by over-expressing the ion transporter β-ENaC in a transgenic mouse, thereby validating the relevance of this to disease pathology. This model has great therapeutic significance but to measure the effect of a small molecule therapy has previously required endpoint analyses on experimental animals. Lung pathology in β-ENaC transgenics is well studied and a number of secondary inflammatory responses are widely involved in early stage disease (and in particular IL upregulation). With this prior knowledge, it is possible to apply the teaching of the present invention to choose a specific promoter that is upregulated due to localised inflammation, engineer this into our lentiviral cassette and produce β-ENaC/promoter-bioimaging somatotransgenics. These animals can subsequently be used as an assay system for drug validation with downstream activity acting as a quantitative assay for pathological progression.

### Example 9

As another example, pathological lung infections with PIV/RSV and Influenza virus (IV) result in early goblet cell hyperplasia/metaplasia and overexpression of mucin genes such as Muc5AC. This early manifestation of virally induced lung disease could be used to follow disease progression or therapeutic regression. Using somatotransgenic bioimaging we will be able to target lung cell types and potentially lung stem cells. Consequently, it will be possible to follow Muc5AC expression in vivo in response to disease states or drug therapies or a combination of both viral or bacterial infection in a disease model such as the β-ENaC transgenics.

### Example 10

The therapeutic reduction of progressive liver/lung fibrosis in diseases such as pulmonary fibrosis, parainfluenza virus (lung) and Hepatitis C (liver) infections would benefit from somatotransgenic bioimaging. Genetically regulating bioimaging reporter output under the control of early effectors activated by TGF-β signalling or downstream markers such as the collagen lag promoter would provide invaluable data on disease progression/regression. TGF-β signalling is integral in early fibrosis in many organs including the liver and lung. Signalling is mediated through downstream Smad signalling which control both pro- and anti-fibrotic responses as well as Epithelial Mesenchymal Transition (EMT) which is implicated in fibrosis as well as other pathologies. Receptor Smads (R-Smads) perpetuate signalling from a stimulated receptor and then co-activate the Effector Smad4 which translocates to the nucleus and initiates transcriptional activation Tnhibitor Smads are known to block this pathway by both binding R-Smad complexes and also at the transcriptional level. This complete process can be followed using somatotransgenics containing promoter/enhancer elements from each stage of this pathway. Furthermore, EMT is controlled by different R-Smads with contrasting downstream effects which can again be modelled and followed in vivo over time. EMT has implications in disparate pathologies such as fibrosis and cancer. Collagen lα2 deposition is characteristic of liver fibrosis and an excellent prognostic marker. Somatotransgenics could subsequently be subjected to liver injury either chemically (CCl₄) or surgically (bile duct ligation) and therapeutics tested in this context with luciferase bioimaging as the output. The ability to image before and after injury as well as before and after treatment highlights the continuity of this process.

1. A method for determining whether the expression of a reporter gene is modulated by a compound, said method comprising:
(a) administering said compound to a non-human transgenic animal, generated by gene transduction of one or more specific tissues when in utero or neonatal, with a vector comprising a bioluminescent reporter gene operably linked to a genetic element responsive to a pathology or therapy; and
(b) determining the effect, if any, of said compound on the expression of said reporter gene in said specific tissue or tissues, said determination comprising detecting from the animal bioluminescence caused by the activity of the gene product of the reporter gene.

2. The method according to claim 1 wherein the vector is a viral vector.

3. The method according to claim 2 wherein the viral vector is an adenoviral, lentiviral, adeno-associated viral (AAV) retroviral vector or herpes simplex virus vector.

4. The method according to claim 1, wherein gene transduction of the in utero or neonatal animal with the vector comprises:
(a) obtaining a vector comprising a bioluminescent reporter gene operably linked to a genetic element responsive to a disease or therapy; and
(b) delivering said vector to one or more selected tissues in a foetal or neonatal animal.

5. The method according to claim 4, wherein delivering said vector is by an injection that targets the vector to one or more specific tissues of said animal.

6. The method according to claim 5, wherein the injection is systemic but the vector is delivered to one or more specific tissues; or wherein the injection is intramuscular, intrathoracic, supracostal, intraperitoneal or intracranial injection.

7. The method according to claim 5, wherein the injection is intravascular and, where the animal is a foetal animal, injection is systemic and the vector is targeted via the yolk sac vessels; or, where the animal is a neonatal animal, the injection is via the superficial temporal vein.

8. The method according to claim 2, wherein the viral vector comprises a tissue-targeting glycoprotein coat.

9. The method according to claim 1, wherein said specific tissue or tissues is/are liver, heart, kidney, muscle, brain, thyroid, lung, pancreas, blood, spleen, thymus, testis, gut, trachea, vascular system, peripheral nervous system or eye tissues.

10. The method according to claim 1, wherein the transgenic animal is a model of a disease and/or contains a knock-out of a gene; or wherein the animal has, or can be made to have, inflammation in the lung, liver, joints, muscle, heart, muscle, brain or other organs.

11. The method according to claim 1, wherein the transgenic animal is a mammal.

12. The method according to claim 11, wherein the mammal is a rodent or primate.

13. The method according to claim 11, wherein the mammal is a mouse, rat, rabbit or mini-pig.

14. The method according to claim 1, wherein the pathology is induced before the administration of said compound.

15. The method according to claim 14, wherein the pathology is induced chemically and/or by surgery.

16. The method according to claim 14, wherein the expression of the reporter gene is detected before and/or after induction of the pathology.

17. The method according to claim 1, wherein the reporter gene is a luciferase gene.

18. The method according to claim 1, wherein the genetic element responsive to a pathology or therapy is a promoter or an enhancer.

19. The method according to claim 1, wherein the pathology is a pathology of the lung or the liver, or of muscle and/or the peripheral or central nervous system.

20. The method according to claim 19, wherein the lung pathology is selected from respiratory infections, pulmonary fibrosis (PF) asthma and chronic obstructive pulmonary disease (COPD); or the liver disease is selected from liver fibrosis, liver cirrhosis and hepatitis C infection; or the disease of the muscle and/or the nervous system is selected from Duchenne Muscular Dystrophy (DMD), Myotonic Dystrophy (MD), Motor Neuron Disease (MND), Alzheimer's Disease (AD) and Huntingdon's Disease (HD).

21. The method according to claim 20, wherein the respiratory infection is caused by Respiratory Syncytial Virus (RSV), Parainfluenza virus (PIV) or Influenza Virus (IV).

22. The method according to claim 1, wherein the pathology comprises inflammation and said determination of bioluminescence determines the response, if any, of the inflammation to administration of said compound.

23. The method according to claim 22 wherein said inflammation comprises inflammation of lung, liver, joints, muscle, heart, muscle, brain or other organs.

24. The use of a non-human transgenic animal generated by gene transduction of one or more specific tissues when in utero or neonatal, with a vector comprising a bioluminescent reporter gene operably linked to a genetic element responsive to a disease or therapy, for determining whether a compound modulates the expression of said reporter gene, by determining the effect, if any, of said compound on the expression of said reporter gene in said specific tissue or tissues, said determination comprising detecting from the animal bioluminescence caused by the activity of the gene product of the reporter gene.

25. A method of evaluating the metabolism and/or toxicity of a compound comprising:
(a) administering said compound to a non-human transgenic animal, generated by (i) gene transduction of one or more specific tissues when in utero or neonatal, with a vector comprising a bioluminescent reporter gene operably linked to a genetic element responsive to drug metabolism and/or drug toxicity, or (ii) introduction., when in utero or neonatal, of transgenic cells comprising a bioluminescent reporter gene operably linked to a genetic element responsive to drug metabolism and/or drug toxicity; and
(b) determining the effect, if any, of said compound on the expression of said reporter gene (i), in said specific tissue or tissues, or (ii) in said introduced cells or cells derived therefrom, said determination comprising detecting from the animal bioluminescence caused by the activity of the gene product of the reporter gene.

26. The method according to claim 25 wherein the vector is a viral vector

27. The method according to claim 26 wherein the viral vector is a lentiviral or retroviral vector.

28. The method according to claim 25, wherein gene transduction of the in utero or neonatal animal with the vector comprises:
(a) obtaining a vector comprising a bioluminescent reporter gene operably linked to a genetic element responsive to drug metabolism and/or toxicity; and
(b) delivering said vector to one or more selected tissues in a foetal or neonatal animal.

29. The method according to claim 28, wherein delivering said vector is by an injection that targets the vector to one or more specific tissues of said animal.

30. The method according to claim 29, wherein the injection is systemic but the vector is delivered to one or more specific tissues.

31. The method according to claim 29, wherein the injection is intravascular and, where the animal is a foetal animal, injection is systemic and the vector is targeted via the yolk sac vessels; or, where the animal is a neonatal animal, the injection is via the superficial temporal vein.

32. The method according to claim 25, wherein said specific tissue or tissues is/are liver tissues.

33. (canceled)

34. The method according to claim 25 wherein said cells are obtained by:
(a) obtaining a vector comprising a bioluminescent reporter gene operably linked to a genetic element responsive to drug metabolism and/or toxicity; and
(b) delivering said vector to said cells.

35. The method according to claim 25 wherein said cells are of foetal, neonatal or adult origin.

36. The method according to claim 25, wherein the cells are cells of an animal of the same species as the animal into which they are introduced or are introduced into the same tissue or organ from which they themselves originate; or are of human origin and are introduced into a compatible non-human animal.

37. The method according to claim 25, wherein the cells are hepatocytes.

38. The method according to claim 37, wherein the hepatocytes are introduced into the liver of the animal.

39. The method according to claim 25, wherein the transgenic animal is a mammal.

40. The method according to claim 39, wherein the mammal is a rodent or primate.

41. The method according to claim 39, wherein the mammal is a mouse, rat, rabbit or mini-pig.

42. The method according to claim 25, wherein the expression of the reporter gene is detected before and after the administration of the compound.

43. The method according to claim 25, wherein the reporter gene is a luciferase gene.

44. The method according to claim 25, wherein the genetic element responsive to drug metabolism and/or drug toxicity is a promoter or an enhancer.

45. The method according to claim 44, wherein the promoter is a cytochrome P450 (CYP450) promoter or the promoter of a gene associated with cytochrome P450 activity.

46. The method according to claim 25, wherein the transgenic animal is a humanised model and/or a disease model and/or contains a knock-out of a gene.

47. The use of a non-human transgenic animal generated by (i) gene transduction of one or more specific tissues when in utero or neonatal, with a vector comprising a bioluminescent reporter gene operably linked to a genetic element responsive to drug metabolism and/or drug toxicity, or (ii) introduction, when in utero or neonatal, of transgenic cells comprising a bioluminescent reporter gene operably linked to a genetic element responsive to drug metabolism and/or drug toxicity,
for determining whether a compound modulates the expression of said reporter gene, by determining the effect, if any, of said compound on the expression of said reporter gene (i) in said specific tissue or tissues, or (ii) in said introduced cells or cells derived therefrom,
said determination comprising detecting from the animal bioluminescence caused by the activity of the gene product of the reporter gene.

48. (canceled)
